(19) United States
(12) Patent Application Publication
(10) Pub. No.: US 2010/0096376 A1
(43) Pub. Date: Apr. 22, 2010

(54) MUSIC GENERATING AND AROMATIC SUBSTANCE RETAINING AND HEATING SYSTEM

(76) Inventor: Ming Jen Hsiao, Toufen Township (TW)

Correspondence Address: HDLS Patent & Trademark Services, P.O. BOX 220746, CHANTILLY, VA, 20153-0746, US

(21) Appl. No.: 12/253,977

(22) Filed: Oct. 19, 2008

(51) Int. Cl. A61L 9/02

(52) U.S. Cl. 219/201; 392/387

(57) ABSTRACT

A music generating and aromatic substance retaining and heating system includes an aromatic substance, and a combination structure of music generator and heater, which includes a housing holding the aromatic substance on the top side, a rectifier transformer disposed outside the housing for converting AC into DC, a music controller disposed outside the housing and electrically connected to the rectifier transformer for outputting a music signal, a heating control circuit board mounted inside the housing and electrically connected to the music controller for controlling a speaker to output music and an electronic heater to heat the aromatic substance to release a scent.

### FIELD OF THE INVENTION

The present invention relates to scent releasing devices and more specifically, to a system for generating music and retraining and heating an aromatic substance.

### DESCRIPTION OF THE RELATED ART

A conventional electronic essential oil heater (the so-called aroma lamp) is known comprising a housing and an electronic heater. The housing holds an essential oil on its top side. The electronic heater is mounted inside the housing. When an electric current is connected to the electronic heater, a heat energy is produced by the electronic heater and transferred to the essential oil, causing the essential oil to release a pleasant smell. This type of electronic essential oil heater is simply to provide a pleasant smell. This single function cannot satisfy modern consumer demands.

Further, U.S. Pat. No. 7,133,605 B2 discloses a device for heating a scented candle, entitled “Heater for scented candles”, for releasing scent in the absence of an open flame. The device includes a housing for retaining the candle. The housing is fabricated from a material having a low thermal conductivity. The device further includes an electrical heater in thermal communication with the housing. The heater warms the candle, and the low thermal conductivity housing aids in retaining heat in the candle thereby causing scent to be released therefrom. Similar to the aforesaid conventional electronic essential oil heater (aroma lamp), this design of heater for scented candles is simply designed to release a pleasant smell in the absence of an open flame.

It is well known that music can be used as a powerful tool for relaxation and stress relief. However, according to conventional designs for retaining and heating a scented candle, it is not practical to use a music generating device with an electronic heater in a housing. Under a high temperature environment, a music generating device cannot function well. Further, according to conventional designs, it is difficult to secure component parts to a ceramic housing.

Therefore, it is desirable to provide a system for retaining and heating an aromatic substance that eliminates the aforesaid drawbacks.

### SUMMARY OF THE INVENTION

The present invention has been accomplished under the circumstances in view. It is one object of the present invention to provide a music generating and aromatic substance retaining and heating system, which has a housing, a rectifier transformer and a music controller be separately arranged to provide an excellent aroma heating effect and a music service, assuring a high level of use and normal LED indication. It is another object of the present invention to provide music generating and aromatic substance retaining and heating system, which has the advantages of ease of installation, ease of control, long service life, and low manufacturing cost. It is still another object of the present invention to provide music generating and aromatic substance retaining and heating system, which has music playing and resistor heating controls be separately arranged so that music play and aroma heating are conveniently performed at a high level of safety.

To achieve these and other objects of the present invention, thee music generating and aromatic substance retaining and heating system comprises an aromatic substance, for example, essential oil, scented candle, scented wax or scented dried flower, and a combination structure of music generator and heater. The combination structure of music generator and heater comprises a housing, a rectifier transformer, a music controller, a heating control circuit board, a speaker, an electronic heater and a fastener. The fastener is to overcome the problem of securing component parts to a ceramic housing. The housing is comprised of a body and a bottom cover. The body is made from ceramic, glass, or any suitable low thermal conductivity material. The body comprises a hollow cylindrical peripheral wall and a top wall connected to the top side of the hollow cylindrical peripheral wall. The top wall defines a top holding space for holding the aromatic substance. The bottom cover closes the bottom side of the hollow cylindrical peripheral wall, thereby defining with the body an inside accommodation chamber. The holding space may be designed in communication with the inside accommodation chamber. In this case, a tray may be detachably mounted in the holding space to hold the aromatic substance. The rectifier transformer has an input terminal and an output terminal. The input terminal is connectable to an AC power source to convert AC into DC for output through the output terminal. The music controller has an input end and an output end. The input end is for the connection of the output terminal of the rectifier transformer. The output end is for outputting a music signal. The heating control circuit board is mounted in the inside accommodation chamber inside the housing, having an electric wire extended to the outside of the bottom cover and an electric socket mounted on the bottom cover outside the housing and electrically connected with the electric wire for the connection of the output end of the music controller. The speaker is mounted on the bottom cover inside the inside accommodation chamber, and electrically connected to the heating control circuit board to receive music signal from the music controller for output. The electronic heater is mounted in the inside accommodation chamber and electrically connected to the heating control circuit board to heat the holding space to a predetermined temperature, causing the aromatic substance to release a scent.

Preferably, the rectifier transformer is mounted in an electric power plug outside the ceramic housing, and therefore no any heat insulative means is necessary in the ceramic housing, facilitating the fabrication.

Preferably, the electronic heater is provided with conducting pins for directly connection to the control circuit board by means of plugging, facilitating the installation and eliminating the drawback of solder bonding that is harmful to environment protection.

Further, by means of the fastener, the electronic heater, the heating control circuit board and the tray are firmly secured together, facilitating the installation.

### BRIEF DESCRIPTION OF THE DRAWINGS

FIG. 1 is a schematic exploded view, partially in sectional elevation, of a system for generating music and retraining and heating an aromatic substance in accordance with a first embodiment of the present invention.

FIG. 2 is another schematic exploded view, partially in sectional elevation, of the system for generating music and retraining and heating an aromatic substance in accordance with the first embodiment of the present invention.

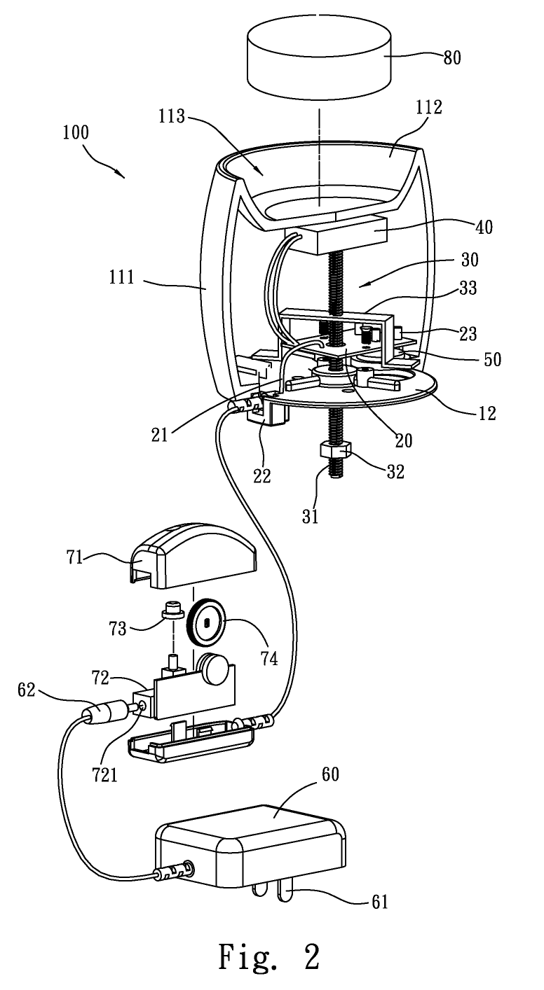

FIG. 3 is a schematic elevational assembly view, partially in section, of the system for generating music and retraining and heating an aromatic substance in accordance with the first embodiment of the present invention.

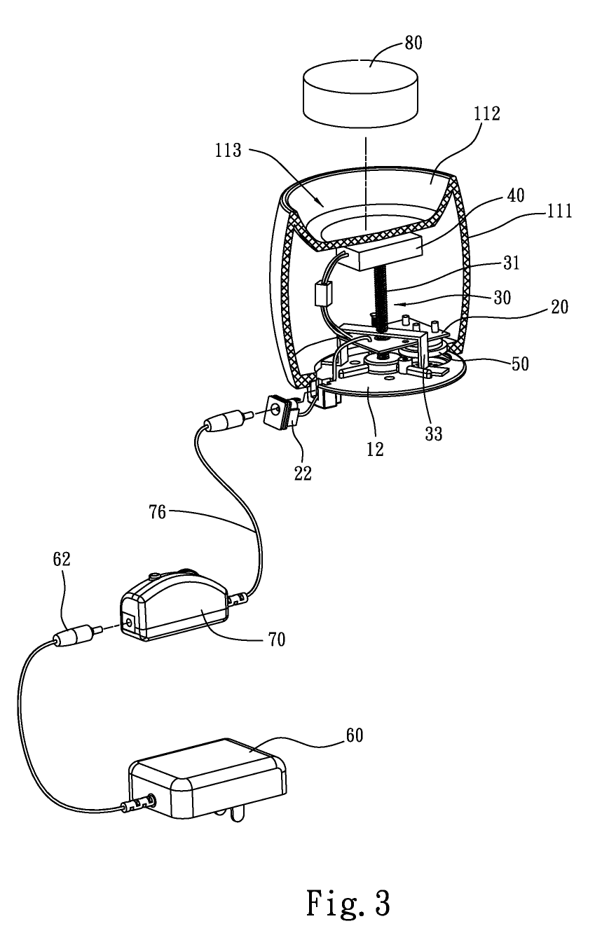

FIG. 4 is a circuit block diagram of the system for generating music and retraining and heating an aromatic substance in accordance with the first embodiment of the present invention.

FIG. 5 is a schematic exploded view, partially in sectional elevation, of a system for generating music and retraining and heating an aromatic substance in accordance with a second embodiment of the present invention.

FIG. 6 is a circuit diagram of the system for generating music and retraining and heating an aromatic substance in accordance with the second embodiment of the present invention.

### DETAILED DESCRIPTION OF THE PREFERRED EMBODIMENT

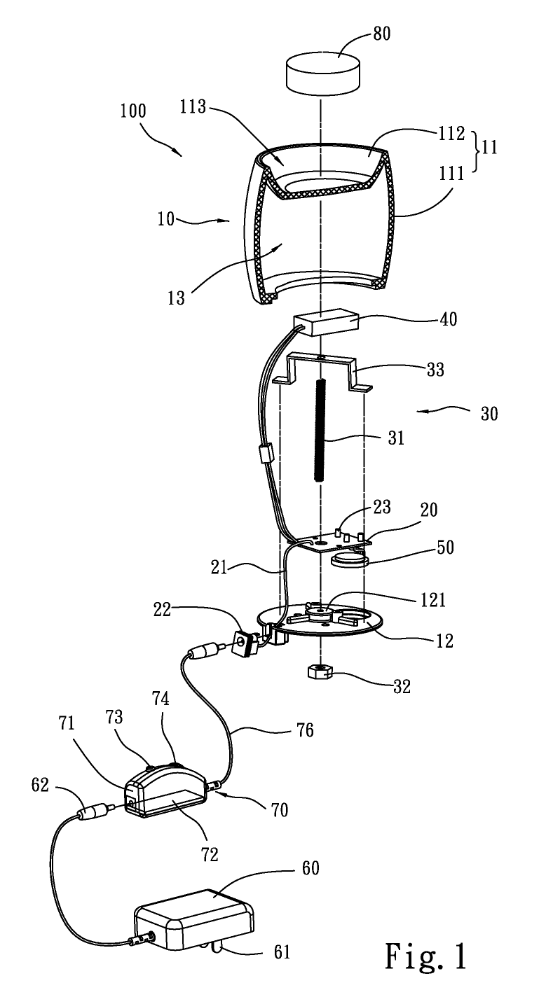

Referring to FIGS. 1˜4, a system for generating music and retraining and heating an aromatic substance in accordance with a first embodiment of the present invention is shown comprising an aromatic substance 80, a combination structure of music generator and heater 100.

The aromatic substance 80 can be an essential oil, scented candle, scented wax, scented dried flower, or any solid or liquid substance that release a pleasant smell when heated.

The combination structure of music generator and heater 100 comprises a housing 10, a heating control circuit board 20, a fastener 30, an electronic heater 40, a speaker 50, a rectifier transformer 60, and a music controller 70.

The housing 10 comprises a body 11 and a bottom cover 12. Further, the housing 10 is made from a low thermal conductivity material, such as ceramic, glass or their compound materials. The body 11 has a hollow cylindrical peripheral wall 111, and a top wall 112 connected to one side, namely, the top side of the hollow cylindrical peripheral wall 111. The top wall 112 has its top surface curved inwards, defining a holding space 113 for holding the aromatic substance 80. The bottom cover 12 is fastened to the other side, namely, the bottom side of the hollow cylindrical peripheral wall 111 to close the hollow cylindrical peripheral wall 111, and therefore the bottom cover 12 defines with the cylindrical peripheral wall 111 an inside accommodation chamber 13. Further, the bottom cover 12 has a through hole 121.

The heating control circuit board 20 is fixedly mounted on the bottom cover 12 of the housing 10 and disposed in the inside accommodation chamber 13 for controlling the operation of the electronic heater 40. Further, an electric socket 22 is fixedly mounted on the bottom cover 12 on the outside of the housing 10 and electrically connected to the heating control circuit board 20 through an electric wire 21. The heating control circuit board 20 has installed therein at least one LED indicator 23 for the indication of the operation status of the related units.

The fastener 30 is comprised of a support rod 31, a lock member 32, and a bracket 33. The support rod 31 according to this embodiment is a screw rod vertically mounted in the inside accommodation chamber 13 inside the housing 10 with its one end extended through the through hole 121 of the bottom cover 12 to the outside of the housing 10. The lock member 32 according to this embodiment is a locknut threaded onto the support rod 31 and stopped outside the bottom cover 12 to lock the support rod 31 to the bottom cover 12. The bracket 33 is mounted in the inside accommodation chamber 13 inside the housing 10 and fastened to the other end of the support rod 31 to stabilize the connection between the support rod 31 and the bottom cover 12.

The electronic heater 40 can be, for example, a heating resistor, preferably, a cement resistor. The electronic heater 40 is electrically connected to the heating control circuit board 20. When an electric current is connected to the electronic heater 40, the electronic heater 40 is caused to produce a thermal energy at a predetermined temperature. The electronic heater 40 can be mounted on the bracket 33 or support rod 31 of the fastener 30, and kept in close contact with the bottom wall of the inside accommodation chamber 13 of the housing 10 for enabling the produced heat energy to be transferred to the bottom wall of the inside accommodation chamber 13.

The speaker 50 is adapted to receive a specific signal and to produce a specific sound subject to the specific signal received. The speaker 50 is mounted the bottom cover 12 of the housing 10 and disposed in the inside accommodation chamber 13, and electrically connected to the heating control circuit board 20.

The rectifier transformer 60 has an input terminal 61 and an output terminal 62. The input terminal 61 is connectable to a city power outlet (not shown) for converting AC into DC for output through the output terminal 62.

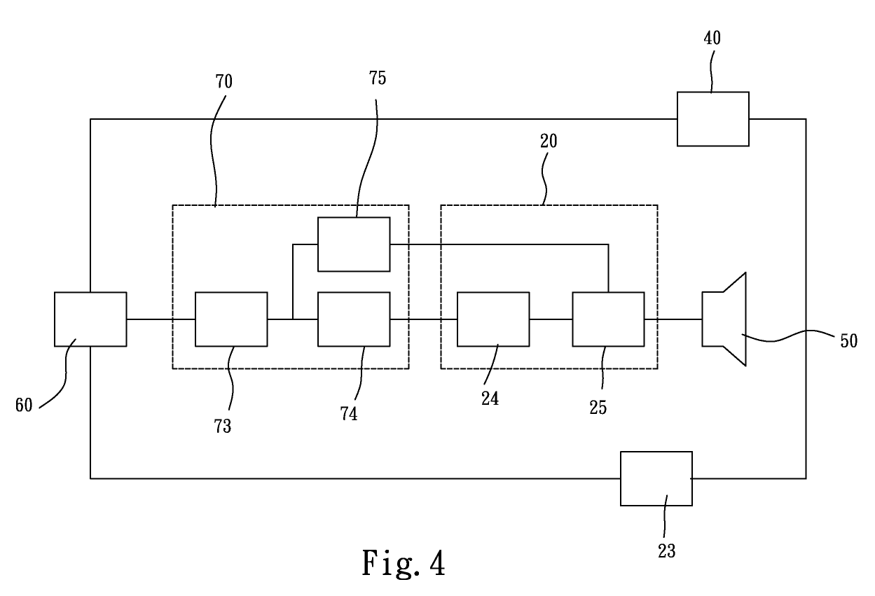

The music controller 70 comprises a casing 71, a music control circuit board 72, a control switch 73, an adjustment wheel 74, a timer 75 (see also FIG. 4), and a cable 76. The music control circuit board 72 is fixedly mounted inside the casing 71 and adapted to output a specific music signal. Further, the music control circuit board 72 has an input terminal 721 and an output terminal (not shown). The input terminal 721 is for the connection of the output terminal 62 of the rectifier transformer 60 to receive from the rectifier transformer 60 the necessary working voltage for the music control circuit board 72. The control switch 73 is electrically connected to the music control circuit board 72, and partially extended out of the casing 71 for operation by a user to switch on/off the music control circuit board 72. The adjustment wheel 74 is electrically connected to the music control circuit board 72, and partially extended out of the casing 71 for operation by a user to regulate the sound volume and to select/store music. The cable 76 has its one end connected to the output terminal 721 of the music control circuit board 72 and its other end connectable to the electric socket 22 of the heating control circuit board 20, enabling the music controller 70 to output DC power supply and music signal to the heating control circuit board 20.

After understanding of the structure of the main parts of the first embodiment of the present invention and their installation and relationship, the operation of the first embodiment of the present invention is explained hereinafter.

When in use, the aromatic substance 80 (scented candle, scented wax, essential oil, or scented dried flower) is put in the holding space 113 of the housing 10, and then the input terminal 61 of the rectifier transformer 60 is connected to a city power supply outlet. Referring also to FIG. 4, the rectifier transformer 60 converts AC power supply into DC power supply and outputs DC power supply to the music control circuit board 72 through the output terminal 62, enabling DC power supply to be directly supplied to the LED indicator 23 and the electronic heater 40. By means of switching on the control switch 73, a music playing action is started. Subject to the control of the adjustment wheel 74, music signal is sent through a circuit 24 and an amplifier 25 to the speaker 50 for output. Further, controlling the setting of the timer 75 can control the operation time of the speaker 50.

As stated above, the invention has the rectifier transformer provided on the outside of the housing for converting AC power supply into DC power supply, avoiding potential danger. Further, the invention has the music controller and the heating control circuit board separately installed and the music controller provided outside the housing, protecting the music controller against potential damage from heat. Therefore, the heating control circuit board can concentrate on heating control, avoiding working temperature control failure.

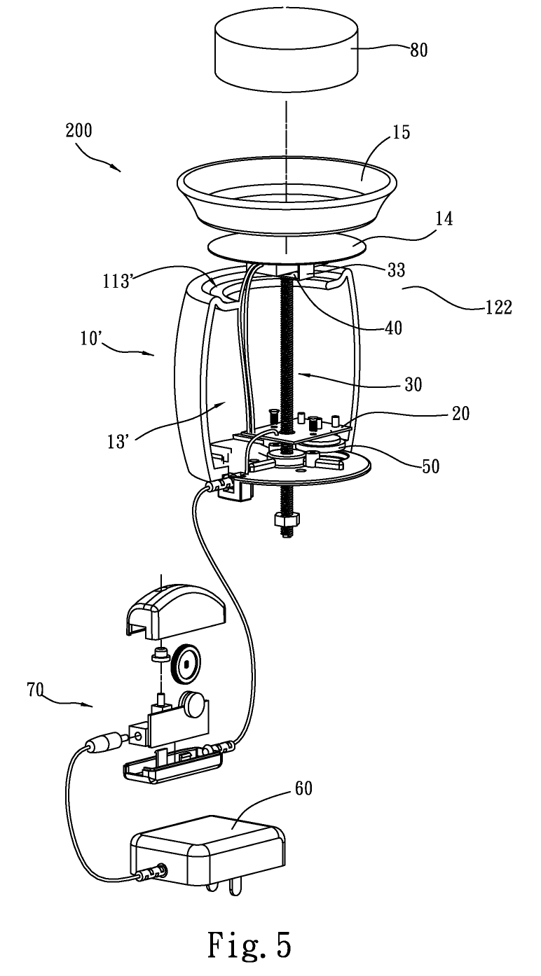

FIG. 5 illustrates a system for generating music and retraining and heating an aromatic substance in accordance with a second embodiment of the present invention. According to this second embodiment, the system is comprised of an aromatic substance 80, and a combination structure of music generator and heater 200.

The combination structure of music generator and heater 200 is comprised of a housing 10, a heating control circuit board 20, a fastener 30, an electronic heater 40, a speaker 50, a rectifier transformer 60, and a music controller 70. The component parts of this second embodiment are substantially similar to the aforesaid first embodiment with the exceptions the features described hereinafter.

The holding space 113′ of the body 11′ of the housing 10′ is in communication with the inside accommodation chamber 13′.

The housing 10′ further comprises a heat transfer panel 14 and a tray 15. The heat transfer panel 14 is made of a high thermal conductivity material, for example, metal. The heat transfer panel 14 is mounted in the holding space 113′ of the body 11′. The electronic heater 40 is mounted on the bracket 33 of the fastener 30 to support the heat transfer panel 14, enabling the heat transfer panel 14 to transfer heat from the electronic heater 40. The tray 15 is made from a low thermal conductivity material, and detachably mounted in the holding space 113′ of the body 11′ in close contact with the heat transfer panel 14 for carrying the aromatic substance 80 so that the aromatic substance 80 can be heated to release a pleasant smell. Because the tray 15 is detachably mounted in the holding space 113′ of the body 11′, it can be conveniently removed from the body 11′ for cleaning.

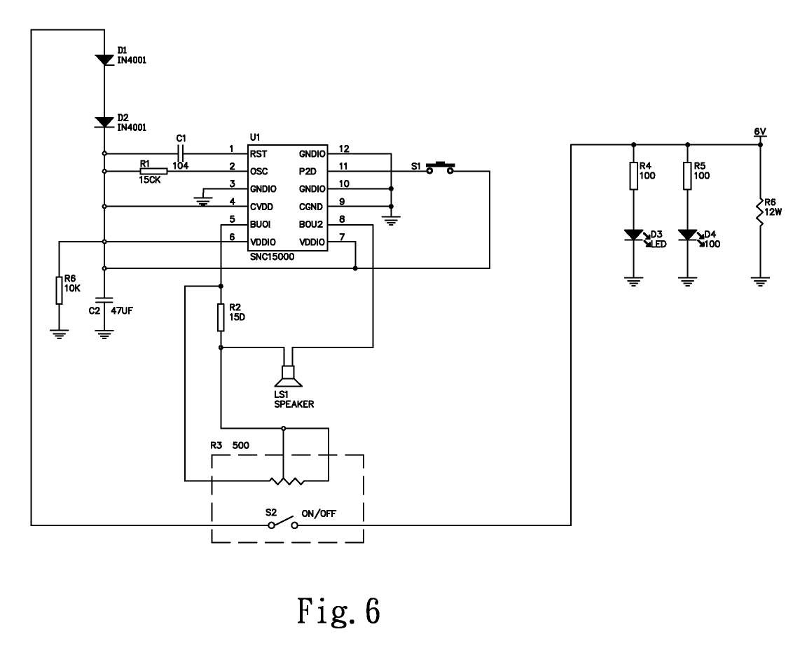

FIG. 6 is a circuit diagram of the aforesaid second embodiment of the present invention. As illustrated, the LED indicator and the electronic heater are at the right half of the loop (in the direction based on FIG. 6), and directly turned to give a visual indication or to emit heat when the control switch is switched on. The left half of the loop is the music play menu, providing volume control, music selection and music storage functions. As illustrated, the music play function and the heating function are disposed at different areas in the loop and independently operated, avoiding interference and facilitating smooth performance of music play function and rapid and stable heating operation.

Although particular embodiments of the invention have been described in detail for purposes of illustration, various modifications and enhancements may be made without departing from the spirit and scope of the invention. Accordingly, the invention is not to be limited except as by the appended claims.

What the invention claimed is:

1. A music generating and aromatic substance retaining and heating system, comprising: an aromatic substance and a combination structure of music generator and heater, said combination structure of music generator and heater comprising:
a housing, said housing comprising a body and a bottom cover, said body being made from a low thermal conductivity material, said body comprising a hollow cylindrical peripheral wall and a top wall connected to a top side of said hollow cylindrical peripheral wall, said top wall defining a holding space in a top side thereof for holding said aromatic substance, said bottom cover being fastened to a bottom side of said hollow cylindrical peripheral wall opposite to said top wall and defining with said cylindrical peripheral wall an inside accommodation chamber;
a rectifier transformer, said rectifier transformer comprising an input terminal and an output terminal, said input terminal being adapted to convert an AC power supply into a DC power supply for output through said output terminal;
a music controller, said music controller comprising an input end electrically connected to the output terminal of said rectifier transformer for receiving a DC power supply from said rectifier transformer, and an output end for outputting a music signal;
a heating control circuit board mounted in said inside accommodation chamber inside said housing, said heating control circuit board comprising an electric wire extending to the outside of said bottom cover and an electric socket mounted on the outside of said bottom cover and electrically connected with said electric wire for the connection of the output end of said music controller to receive a music signal from said music controller and the DC power supply being received by said music controller from said rectifier transformer;
a speaker mounted in said bottom cover of said housing and electrically connected to said heating control circuit board and adapted to receive a music signal from said music controller for output; and
a fastener connected to said bottom cover to hold said electronic heater in place.

2. The music generating and aromatic substance retaining and heating system as claimed in claim 1, wherein said aromatic substance is an essential oil.

3. The music generating and aromatic substance retaining and heating system as claimed in claim 1, wherein said aromatic substance is selected from a group of scented candle, scented wax and scented dried flower.

4. The music generating and aromatic substance retaining and heating system as claimed in claim 1, wherein said body of said housing is prepared from a material group of ceramic and glass.

5. The music generating and aromatic substance retaining and heating system as claimed in claim 1, wherein said heating control circuit board comprises at least one LED indicator means.

6. The music generating and aromatic substance retaining and heating system as claimed in claim 1, wherein said fastener comprises a support rod, a lock member and a bracket, said support rod being vertically mounted in said inside accommodation chamber inside said housing, said support rod having a bottom end extended out of said bottom cover and a top end opposite to said bottom end, said lock member being fastened to said support rod to lock said support rod to said bottom cover, said bracket being mounted in said inside accommodation chamber inside said housing and fastened to the top end of said support rod.

7. The music generating and aromatic substance retaining and heating system as claimed in claim 6, wherein said support rod is a screw rod; said lock member is a locknut; said bottom cover comprises a through hole for the passing of the bottom end of said support rod.

8. The music generating and aromatic substance retaining and heating system as claimed in claim 6, wherein said holding space of said top wall of said body of said housing is disposed in communication with said inside accommodation chamber; said housing further comprises a heat transfer panel mounted in said holding space of said housing and supported on said bracket for transferring heat energy from said electronic heater, and a tray detachably mounted in said holding space and kept in contact with said heat transfer panel for holding and heating said aromatic substance to release a scent.

9. The music generating and aromatic substance retaining and heating system as claimed in claim 1, wherein said electronic heater is a heating resistor.

10. The music generating and aromatic substance retaining and heating system as claimed in claim 1, wherein said music controller comprises a casing, a music control circuit board, a control switch, an adjustment wheel, a timer, and a cable, said music control circuit board being fixedly mounted inside said casing and adapted to output a music signal, said music control circuit board comprising an input terminal and an output terminal, the input terminal of said music control circuit board being electrically connected to the output terminal of said rectifier transformer to receive from said rectifier transformer the necessary working voltage for said music control circuit board, said control switch being electrically connected to said music control circuit board and partially extended out of said casing for operation by a user to switch on/off said music control circuit board, said adjustment wheel being electrically connected to said music control circuit board and partially extended out of said casing for operation by a user to regulate sound volume and to select/store music, said cable having one end thereof electrically connected to the output terminal of said music control circuit board and an opposite end thereof connectable to said electric socket of said heating control circuit board for enabling said music controller to output a DC power supply and a music signal to said heating control circuit board.

11. The music generating and aromatic substance retaining and heating system as claimed in claim 1, wherein said holding space of said housing is in communication with said inside accommodation chamber; said housing further comprises a heat transfer panel and a tray, said heat transfer panel being mounted in said holding space of said housing and adapted to transfer heat from said electronic heater, said tray being detachably mounted in said holding space of said housing and kept in contact with said heat transfer panel for holding and heating said aromatic substance to release a scent.

12. The music generating and aromatic substance retaining and heating system as claimed in claim 11, wherein said holding tray is made from a low thermal conductivity material.

13. The music generating and aromatic substance retaining and heating system as claimed in claim 1, wherein said rectifier transformer and said music controller are arranged outside said housing.

14. A music generating and aromatic substance retaining and heating system, comprising:
a housing, said housing comprising a body and a bottom cover, said body being made from a low thermal conductivity material, said body comprising a hollow cylindrical peripheral wall and a top wall connected to a top side of said hollow cylindrical peripheral wall, said top wall defining a holding space in a top side thereof, said bottom cover being fastened to a bottom side of said hollow cylindrical peripheral wall opposite to said top wall and defining with said cylindrical peripheral wall an inside accommodation chamber;
a rectifier transformer, said rectifier transformer comprising an input terminal and an output terminal, said input terminal being adapted to convert an AC power supply into a DC power supply for output through said output terminal;
a music controller, said music controller comprising an input end electrically connected to the output terminal of said rectifier transformer for receiving a DC power supply from said rectifier transformer, and an output end for outputting a music signal;
a heating control circuit board mounted in said inside accommodation chamber inside said housing and electrically connected to said music controller;
a speaker mounted in said bottom cover of said housing and disposed inside said inside accommodation chamber and electrically connected to said heating control circuit board and adapted to receive a music signal from said music controller for output; and
an electronic heater mounted in said inside accommodation chamber and electrically connected to said heating control circuit board for heating said holding space.

15. The music generating and aromatic substance retaining and heating system as claimed in claim 14, wherein said body of said housing is prepared from a material group of ceramic and glass.

16. The music generating and aromatic substance retaining and heating system as claimed in claim 14, wherein said heating control circuit board comprises an electric wire extending to the outside of said bottom cover and an electric socket mounted on the outside of said bottom cover and electrically connected with said electric wire for the connection of the output end of said music controller.

17. The music generating and aromatic substance retaining and heating system as claimed in claim 14, wherein said heating control circuit board comprises at least one LED indicator means.

18. The music generating and aromatic substance retaining and heating system as claimed in claim 14, further comprising a fastener, said fastener comprises a screw rod, a locknut and a bracket, said screw rod being vertically mounted in said inside accommodation chamber, said screw rod having a bottom end extending out of a through hole of said bottom cover and a top end suspending in said inside accommodation chamber, said locknut being fastened to said screw rod to lock said screw rod to said bottom cover, said bracket being mounted in said inside accommodation chamber inside said housing and fastened to the top end of said screw rod to hold said electronic heater in place.

19. The music generating and aromatic substance retaining and heating system as claimed in claim 14, wherein said music controller comprises a casing, a music control circuit board, a control switch, an adjustment wheel, a timer, and a cable, said music control circuit board being fixedly mounted inside said casing and adapted to output a music signal, said music control circuit board comprising an input terminal and an output terminal, the input terminal of said music control circuit board being electrically connected to the output terminal of said rectifier transformer to receive from said rectifier transformer the necessary working voltage for said music control circuit board, said control switch being electrically connected to said music control circuit board and partially extended out of said casing for operation by a user to switch on/off said music control circuit board, said adjustment wheel being electrically connected to said music control circuit board and partially extended out of said casing for operation by a user to regulate sound volume and to select/store music, said cable having one end thereof electrically connected to the output terminal of said music control circuit board and an opposite end thereof connectable to said electric socket of said heating control circuit board for enabling said music controller to output a DC power supply and a music signal to said heating control circuit board.

20. The music generating and aromatic substance retaining and heating system as claimed in claim 14, wherein said holding space of said top wall of said body of said housing is disposed in communication with said inside accommodation chamber; said housing further comprises a heat transfer panel made from a high thermal conductivity material and mounted in said holding space of said housing and supported on said bracket for transferring heat energy from said electronic heater, and a tray made from a low thermal conductivity material and detachably mounted in said holding space and kept in contact with said heat transfer panel for holding and heating an aromatic substance to release a scent.
